(19) United States
(12) Patent Application Publication
(10) Pub. No.: US 2004/0192781 A1
(43) Pub. Date: Sep. 30, 2004

(54) Method of administration for metoclopramide and pharmaceutical formulation therefor

(76) Inventor: Eugene T. Haley, Wilmington, NC (US)

Correspondence Address: Eugene T. Haley, 317 Bradley Creek Point Road, Wilmington, NC, 28403, US

(21) Appl. No.: 10/401,389

(22) Filed: Mar. 31, 2003

(51) Int. Cl. A61K 31/165

(52) U.S. Cl. 514/620

(57) ABSTRACT

A method for abating manifestations of gastroparesis in a patient, including administering an immediate release formulation of metoclopramide to a patient prior to the patient performing an event known to cause gastroparesis in that patient. The administration of the metoclopramide in this manner abates the rapid onset of gastroparesis in the patient after the patient performs the event that normally causes gastroparesis. A pharmaceutical dosage of metoclopramide having an oral immediate release formulation effective for obtaining a Tₘₐₓ for metoclopramide of from about less than one hour with an amount of metoclopramide effective for abating the rapid onset of gastroparesis.

### BACKGROUND OF THE INVENTION

1. Field of the Invention

The present invention provides an oral immediate release metoclopramide formulation and administration of metoclopramide in an oral immediate release formulation to abate the rapid onset of gastroparesis.

2. Brief Description of the Related Art

Gastroparesis occurs when the stomach fails to properly empty because of decreased gastric motility. Normally, the stomach contracts slowly to squeeze solid food into small compressed particles, that are pushed into the small bowel, however, decreased gasric motility leads to non-movement of the food, resulting in the food remaining in the stomach longer. As food lingers too long in the stomach, problems of bacterial overgrowth from the fermentation of food or food hardening into solid masses, called bezoars, may occur. Symptoms of gastroparesis may include mild or severe nausea, vomiting, abdominal discomfort and bloating, feeling of immediate fullness upon eating, and loss of appetite. Dehydration and stomach bleeding, and other medical problems may occur, as well as obstruction in the stomach such as blockage of the passage of food into the small intestine.

Metoclopramide, chemically known as 4-amino-5-chloro-2-methoxy-N-[(2-diethyl-amino)ethyl]benzamide, is used to treat gastroparesis, by stimulating stomach activity to empty the stomach. This additionally may provide relief from nausea and vomiting. Commercially in the United States, metoclopramide is marketed under the tradename Reglan™. Reglan™ is a pharmaceutical salt of metoclopramide, metoclopramide hydrochloride, in tablet form.

Metoclopramide has been disclosed in several formulations. In U.S. Pat. No. 4,309,408 to Pathak et al., entitled “Effervescent Powders”, effervescent powders containing a certain paracetamol and metoclopramide or an acid addition salt thereof is disclosed. U.S. Pat. No. 6,264,981 to Zhang et al., entitled “Oral Transmucosal Drug Dosage Using Solid Solution”, claims an improved oral transmucosal solid dosage. A nasal immediate release dosage form of metoclopramide has been disclosed in U.S. patent application No. 2002/0065321 to Lehman et al., entitled “Nasal Administration of Agents for the Treatment of Gastroparesis”. Within the 2002/0065321 patent application, problems of the commercially available metoclopramide are detailed as:

Metoclopramide (MCP) stimulates stomach muscle contractions to help empty food. It also helps reduce nausea and vomiting. Metoclopramide is taken 20 to 30 minutes before meals and at bedtime. Traditionally, treatment of gastroparesis is via injection or oral route. Metoclopramide is currently available in a tablet form, injection form, and syrup form under the name Reglan® (A.H. Robbins Company). The injection form has an onset of action of about 1-3 minutes after intravenous administration and an onset of action of about 10-15 minutes after intramuscular administration. However, injections, particularly daily multiple injections, are often very painful and inconvenient. Intravenous administration often requires a hospital setting. As a result, compliance (compliance=following dosage regimen prescribed) is often very poor. Metoclopramide in the tablet or syrup form can be effectively and rapidly absorbed through the GI tract by healthy persons. Pharmacokinetics studies of subjects show that oral bioavailability of metoclopramide is approximately 80%±15.5%. Peak plasma concentrations occur at about 1-2 hours after a single oral dose. However, for patients with gastroparesis, metoclopramide absorption through the GI tract is unpredictable and far less effective, with predictability and effectiveness having an inverse relationship to the severity of the symptom, i.e., the more severe the symptoms, the less likely that oral administration is an option. Further complicating the matter of oral administration of metoclopramide is the fact that patients with gastroparesis often have symptoms such as vomiting and nausea. If vomiting takes place, the amount of metoclopramide that remains in the stomach is unknown, and the result of treatment is even less predictable.

Additionally the 2002/0065321 patent application discloses that nasal “dosages for the treatment and control of gastroparesis are usually given before meals and before bed time.” Problematic with the nasal dosage form of metoclopramide in the 2002/0065321 patent application is patient compliance as nasal sprays have limited patient compliance within certain patient populations, including limitations of use due to allergies or colds.

There is a need in the art to provide a dosage form and administration of metoclopramide that overcomes the disadvantages of current commercial tablet dosages, and that additionally enhances patient compliance relative to nasal and patient convenience to intravenous administration. The present invention addresses these and other needs.

### SUMMARY OF THE INVENTION

The present invention includes a method for abating manifestations of gastroparesis in a patient comprising the steps of administering metoclopramide, or a pharmaceutically acceptable salt thereof, to a patient prior to the patient performing a prescribed gastroparesis causing event, wherein said metoclopramide is in a pharmaceutically acceptable immediate release oral formulation and performing, by the patient, the prescribed gastroparesis causing event, wherein the administration of the immediate release oral formulation abates the rapid onset of gastroparesis in the patient. Most typically the prescribed gastroparesis causing event includes consumption of food.

The present invention also includes a pharmaceutical dosage of metoclopramide comprising an oral immediate release formulation effective for obtaining a Tₘₐₓ for metoclopramide of from about less than one hour and an amount of metoclopramide effective for abating the rapid onset of gastroparesis.

### DETAILED DESCRIPTION OF THE PREFERRED EMBODIMENT

The present invention provides for a novel approach to treating the rapid onset of gastroparesis in a patient. The present invention includes a method for abating manifestations of gastroparesis in a patient by administering metoclopramide, including pharmaceutically acceptable salts thereof, to the patient prior to the patient performing a prescribed gastroparesis causing event. The present invention also includes a pharmaceutical dosage of metoclopramide comprising an oral immediate release formulation effective for obtaining a Tₘₐₓ for metoclopramide of from about thirty minutes or less and an amount of metoclopramide effective for abating the rapid onset of gastroparesis.

By administrating the metoclopramide to the patient prior to the onset of gastroparesis, the effects of the gastroparesis are minimized. With the present invention the effects of gastroparesis are minimized during the first moments of onset, such as for example at the first instance, or the first 10, 15, 20 or 30 minutes. Unlike other oral dosage forms of metoclopramide, the immediate release oral formulation of the present invention addresses the problems of the rapid onset of gastroparesis, and as such, increases patient compliance and effectiveness. Oral dosage forms also increase patient compliance over non-oral formulations, such as nasal or other forms.

The present invention has the distinct advantage of providing a Tₘₐₓ of metoclopramide within from about less than one hour, more preferably from about 45 minutes or less, still more preferably from about 30 minutes or less, and most preferably from about 15 minutes or less, such as for example without limitation from about 10 minutes to about 25 minutes, from about 15 minutes to about 30 minutes, from about 20 to about 40 minutes, etc.

As many patients understand the gastroparesis consequences of certain prescribed gastroparesis causing events that they must partake in, these patients may administer the immediate release oral formulation prior to them performing the prescribed gastroparesis causing event, reducing the discomfort that the patient experiences with the rapid onset of the gastroparesis. With currently known oral formulations of metoclopramide, anticipation of a prescribed gastroparesis causing event occurs significantly before the prescribed gastroparesis causing event to provide effective relief in abating the effects of gastroparesis. For example, with the present invention the patient may take the immediate release formulation once seated to begin the meal, with a Tₘₐₓ occurring for example without limitation from about 10 minutes to about 30 minutes later, i.e., effectively occurring at a simultaneous time or before during the eating process. This creates a routine and regime, particularly with older patients, that greatly increases patient compliance.

Rapid onset of the gastroparesis that occurs from the time the prescribed gastroparesis causing event occurs to the time of the gastroparesis. In the case of the intake of food, for example, gastroparesis may occur from the movement the food is not properly advanced through the stomach. Rapid onset may include, for example without limitation, times of from about forty minutes or less, such as about 0, 5, 10, 15, 20, 25, and 30 minutes, and times and ranges of times therebetween. Abating manifestations of the rapid onset of gastroparesis in a patient includes the elimination, reduction, mitigation or other like degrees of alleviating discomfort and/or medical complications of gastroparesis in a manner that addresses the rapidity of the problem.

Prescribed gastroparesis causing events are those events that can be anticipated by the patient, or other individual responsible for administering metoclopramide, to occur from a given set of circumstances. Such circumstances include, for example without limitation, eating food, such as eating a meal including breakfast, lunch or dinner, laying down to sleep, drinking specific type of liquids, engaging in an exercise routine, engaging in an unpleasant event, such as for example attending a business meeting, school conference and the like. As such prescribed gastroparesis causing events are generally known prior to the rapid onset of gastroparesis in the patient, this allows administration of metoclopramide by the patient prior to the event, either by patient self-dosing or dosing of the patient by another, such as a mother, doctor, etc.

Recurrence of gastroparesis becomes expected for certain individuals as those individuals need to repeat certain tasks in the normal course of their livelihood. In the common prescribed gastroparesis causing event of consumption of food, administration with the preparation of the eating event, such as sitting down to eat, entering a restaurant, etc., aids the person in a compliance routine that is beneficial to taking the drug. In the case of administration at bedtime, the rapid onset of gastroparesis with the act of laying down is abated, as the person laying down is aid in a regimented compliance routine. Additionally, with the occurrence of gastroparesis prior to the administration of the oral immediate release formulation, the present invention decreases the time of discomfort once the formulation is taken.

Patients are defined herein as humans, including men, women, and children, either individuals or groups, awaiting or under medical care and/or treatment. Medical care and/or treatment includes medical care or treatment by a medical professional, such as a doctor or nurse, self-administered treatments, treatment by others, and the like. Additionally, patients include mammals other than humans, including for example without limitation, dogs, cats, and the like. In general, mammals that have previously experienced gastroparesis, or there is reason to believe that mammal would benefit from treatment for gastroparesis, are patients. Patients include a broad segment of the population, and as such, are preferably instructed by qualified persons, such as doctors, nurses, pharmacists, etc., either by prescription or other method, to take metoclopramide as taught herein. Such instruction may include, for example, taking metoclopramide in an immediate release dosage at a given time prior to eating, taking metoclopramide in an immediate release dosage with the first glass of water when eating at a restaurant, etc.

Preferably, the immediate release oral formulation comprises a mouth dissolving formulation that dissolves in the mouth or a rapidly dissolving formulation for the gastrointestinal track. More preferably the formulations that dissolve in the mouth include a dosage form of either a sublingual or buccal delivery system. In one particular embodiment, the formulation comprises a sachet. In an alternative embodiment the immediate release oral formulation comprises a dosage form that dissolves within the digestive system beyond the mouth, e.g., throat, stomach, gastro-intestinal track, etc., using such dosage forms as a tablet, pill or capsule.

Administering the immediate release formulation of metoclopramide may occur in any appropriate time period prior to the onset of gastroparesis, preferably the time period of administering the metoclopramide occurs from about thirty minutes or less prior to the gastroparesis causing event, more preferably from about twenty minutes or less prior to the prescribed gastroparesis causing event, and most preferably from about ten minutes or less, such as from about twenty minutes to about ten minutes, or from about fifteen minutes to about five minutes prior to the prescribed gastroparesis causing event. Administration of the oral immediate release formulation of the present invention also may occur after the onset of gastroparesis.

Metoclopramide is administered in any appropriate dosage amount that mitigates the rapid onset of gastroparesis from a prescribed gastroparesis causing event, such as preferably from about 2 mg to about 240 mg, from about 5 mg to about 160 mg, from about 5 mg to about 80 mg, from about 5 mg to about 40 mg, from about 5 mg to about 20 mg, from about 5 mg to about 10 mg, or other amounts and ranges of amounts that are effective in reducing, mitigating, or otherwise alleviating the effects of gastroparesis as determinable by one skilled in the art in light of the disclosure herein. Such amounts should be administered that are safe for the patient taking the metoclopramide dosage, and accordingly dosages in amounts of metoclopramide of from about 5 mg to about 20 mg, from about 5 mg to about 10 mg are most preferred.

The administration of the immediate release oral formulation, as taught herein, results in an abated gastroparesis event from the rapid onset of gastroparesis in the patient.

The foregoing summary, description, and examples of the present invention are not intended to be limiting, but are only exemplary of the inventive features which are defined in the claims.

What is claimed is:

1. A method for abating manifestations of gastroparesis in a patient comprising the steps of:
administering metoclopramide, or a pharmaceutically acceptable salt thereof, to a patient prior to the patient performing a prescribed gastroparesis causing event, wherein said metoclopramide is in a pharmaceutically acceptable immediate release oral formulation; and,
performing, by the patient, the prescribed gastroparesis causing event, wherein the administration of the immediate release oral formulation abates the rapid onset of gastroparesis in the patient.

2. The method of claim 1, wherein the immediate release oral formulation comprises a formulation that dissolves in the mouth.

3. The method of claim 2, wherein the mouth dissolving formulation comprises a dosage form selected from the group consisting of sublingual and buccal delivery systems.

4. The method of claim 3, wherein the dosage form comprise a sublingual delivery system.

5. The method of claim 3, wherein the dosage form comprise a buccal delivery system.

6. The method of claim 2, wherein the dosage form comprises a sachet.

7. The method of claim 1, wherein the immediate release oral formulation comprises a dosage form selected from the group consisting of tablet, pill, and capsule.

8. The method of claim 1, wherein the immediate release oral formulation creates a Tₘₐₓ for metoclopramide of from about thirty minutes or less.

9. The method of claim 1, wherein the step of administering metoclopramide occurs from about thirty minute or less prior to the prescribed gastroparesis causing event.

10. The method of claim 9, wherein the step of administering metoclopramide occurs from about twenty minutes to about ten minutes prior to the prescribed gastroparesis causing event.

11. The method of claim 1, wherein the prescribed gastroparesis causing event comprises eating food.

12. The method of claim 11, wherein the prescribed gastroparesis causing event comprises eating a meal.

13. The method of claim 1, wherein the administered metoclopramide is present in an amount of from about 5 mg to about 20 mg.

14. The method of claim 13, wherein the administered metoclopramide is present in an amount of from about 5 mg to about 10 mg.

15. An abated gastroparesis event produced by the method of claim 1.

16. A pharmaceutical dosage of metoclopramide comprising an oral immediate release formulation effective for obtaining a Tₘₐₓ for metoclopramide of from about less than one hour and an amount of metoclopramide effective for abating the rapid onset of gastroparesis.

17. The pharmaceutical dosage of claim 16, comprising an oral immediate release formulation effective for obtaining a Tₘₐₓ for metoclopramide of from about 45 minutes or less.

18. The pharmaceutical dosage of claim 16, comprising an oral immediate release formulation effective for obtaining a Tₘₐₓ for metoclopramide of from about 30 minutes or less.

19. The pharmaceutical dosage of claim 16, comprising an oral immediate release formulation effective for obtaining a Tₘₐₓ for metoclopramide of from about 15 minutes or less.

20. Administering to a patient experiencing gastroparesis the pharmaceutical dosage of metoclopramide of claim 19.
